(19) United States
(12) Patent Application Publication
(10) Pub. No.: US 2010/0256497 A1
(43) Pub. Date: Oct. 7, 2010

(54) OSCILLATOR FOR ULTRASONIC WAVE RECEPTION, ITS MANUFACTURING METHOD, ULTRASONIC WAVE PROBE AND ULTRASONIC WAVE MEDICAL DIAGNOSTIC IMAGING SYSTEM

(76) Inventor: Kenji Ohnuma, Tokyo (JP)

Correspondence Address: FRISHAUF, HOLTZ, GOODMAN & CHICK, PC, 220 Fifth Avenue, 16TH Floor, NEW YORK, NY, 10001-7708, US

(21) Appl. No.: 12/742,473

(22) PCT Filed: Oct. 10, 2008
(86) PCT No.: PCT/JP2008/068465

§ 371 Date: May 12, 2010

(30) Foreign Application Priority Data
Nov. 21, 2007 (JP) 2007 301331

(51) Int. Cl. A61B 8/00; H04R 31/00

(52) U.S. Cl. 600/459; 29/594

(57) ABSTRACT

The present invention is to provide an oscillator for ultrasonic wave reception which is excellent in piezoelectric characteristic and thermal resistance and is suitable for high frequency and broad band, a manufacturing method of the oscillator, an ultrasonic wave probe employing the oscillator, and an ultrasonic wave medical diagnostic imaging system. The oscillator for ultrasonic wave reception of the invention is used in a probe for an ultrasonic wave medical diagnostic imaging system, and comprises a piezoelectric material for ultrasonic wave reception, wherein the piezoelectric material for ultrasonic wave reception is an organic piezoelectric material having vinylidene fluoride as a main component, and the organic piezoelectric material has been heat treated and has a relative dielectric constant in a thickness resonance frequency being 10 or more.

### FIELD OF THE INVENTION

The present invention relates to an oscillator for ultrasonic wave reception which is suitable for high frequency and broad band, a manufacturing method of the oscillator, an ultrasonic wave probe employing the oscillator, and an ultrasonic wave medical diagnostic imaging system.

### TECHNICAL BACKGROUND

Generally, a sonic wave of 16,000 Hz or more is collectively called an ultrasonic wave. The ultrasonic wave makes it possible to check the inside of an object without being destroyed and harmlessly and is utilized in various fields such as detection of defects, diagnosis of disease, and others. One of the applications is an ultrasonic wave diagnostic system, in which the inside of an examinee is scanned by an ultrasonic wave to form an image of the inside of the examinee based on a reception signal generated from a reflection ultrasonic wave (echo) from the inside. This ultrasonic wave diagnostic system employs an ultrasonic wave probe which transmits an ultrasonic wave to an examinee and receives an ultrasonic wave from the examinee. This ultrasonic wave probe employs an ultrasonic wave transmission and reception element which is provided with an oscillator which vibrates mechanically based on a transmission signal and generates an ultrasonic wave and produces a reception signal by receiving a reflection ultrasonic wave generated from difference of the acoustic impedance in the inside of the examinee.

In recent years, a harmonic imaging technique has been studied and developed which forms an in-examinee image employing the harmonic frequency component of an ultrasonic wave transmitted from an ultrasonic wave probe to the examinee instead of the frequency (fundamental frequency) component of the transmitted ultrasonic wave. This harmonic imaging technique has many advantages in that (1) the side lobe level is small as compared with that of the fundamental frequency component and the S/N ratio (signal to noise ratio) is improved, resulting in improved contrast resolution, (2) the high frequency reduces the beam width, resulting in improved resolution in the lateral direction, (3) the low sound pressure and small sound pressure fluctuation at a short distance minimizes the multiple reflection, and (4) the attenuation beyond focus, which is at the same level as that of the fundamental wave provides high depth-speed as compared with the case where a high frequency wave is used as the fundamental wave.

The ultrasonic wave probe for the harmonic imaging requires a broad frequency band ranging from the frequency of a fundamental wave to the frequency of a harmonic wave. The frequency region on the lower frequency side is employed for transmission to transmit the fundamental wave, while the frequency region on the higher frequency side is employed for reception to receive the fundamental wave (see for example, Patent Document 1).

The ultrasonic wave probe disclosed in this Patent Document 1 is one which, when applied to an examinee, transmits an ultrasonic wave to the inside of the examinee, and receives an ultrasonic wave returned by reflection therefrom. The ultrasonic wave probe is provided with a first piezoelectric layer composed of a plurality of arranged first piezoelectric elements having a predetermined first acoustic impedance, the first piezoelectric layer transmitting a fundamental wave comprised of an ultrasonic wave with a predetermined center frequency to an examinee and receiving a fundamental wave among the ultrasonic waves returned by reflection from the inside of the examinee. This ultrasonic wave probe is also provided with a second piezoelectric layer composed of a plurality of arranged second piezoelectric elements having a predetermined second acoustic impedance smaller than the predetermined first acoustic impedance, the second piezoelectric layer receiving a harmonic wave among the ultrasonic waves returned by reflection from the inside of the examinee. Herein, the second piezoelectric layer is overlapped on the entire surface of the first piezoelectric layer on the side on which the ultrasonic wave probe is applied to the examinee. The ultrasonic wave probe having the structure as described above can transmit and receive an ultrasonic wave in a broad frequency band.

The fundamental wave in harmonic imaging is preferably a sonic wave having the possible narrowest band width. As a piezoelectric element, so-called ceramics inorganic piezoelectric materials are widely used which include a single crystal of quartz, LiNbO₃, LiTaO₃ or KNbO₃; a thin film of ZnO or AlN; and calcination products of Pb(Zr, Ti)O₃ and the like, each subjected to polarization treatment. However, these inorganic materials are not suitable for application to a piezoelectric element detecting a reception wave on a higher frequency side requiring sensitivity to a broader band region. As a piezoelectric element suitable for high frequency and broad band, an organic piezoelectric material employing an organic polymer material such as polyvinylidene fluoride (hereinafter also referred to as PVDF) is known (see for example, Patent Document 2). This organic piezoelectric material is flexible, and easy to form a thin film, a large area or a long length, as compared with inorganic piezoelectric materials, and therefore, has advantages of manufacturing those in any shape or structure.

However, the element composed of the organic piezoelectric material is low in the phase transition temperature as compared with one composed of the inorganic piezoelectric material, and therefore, has a defect in that thermal resistance is poor. When an organic piezoelectric material is used in an ultrasonic wave probe together with an inorganic piezoelectric material, a countermeasure to heat applied during the manufacture or heat applied in sterilization or disinfection carried out under circumstances employed is required. With respect to improved thermal resistance of PVDF, a vinylidene fluoride copolymer with excellent thermal resistance, obtained according to a manufacturing method which does not provide a phase transition temperature losing a piezoelectric characteristic, is disclosed in the Patent Document 2. The method is effective for a heat melting product of a copolymer of vinylidene fluoride and trifluoroethylene (hereinafter also referred to as 3FE) having a specific composition ratio, however, it is not applied to a copolymer having a different composition ratio or to a stretched film, since they have problem in that the performance deteriorates at high temperature approximate to the film melting point
Patent Document 1: Japanese Patent O.P.I. Publication No. 11-276478
Patent Document 2: Japanese Patent O.P.I. Publication No. 60-217674

### DISCLOSURE OF THE INVENTION

### Problems to be Solved by the Invention

The present invention has been made in view of the above. An object of the invention is to provide an oscillator for ultrasonic wave reception which is excellent in piezoelectric characteristic and thermal resistance and is suitable for high frequency and broad band, a manufacturing method of the oscillator, an ultrasonic wave probe employing the oscillator, and an ultrasonic wave medical diagnostic imaging system.

### Means for Solving the Above Problems

The above object of the invention can be attained by any one of the following constitutions.

[1] An oscillator for ultrasonic wave reception used in a probe for an ultrasonic wave medical diagnostic imaging system, the oscillator for ultrasonic wave reception comprising a piezoelectric material for ultrasonic wave reception, wherein the piezoelectric material for ultrasonic wave reception is an organic piezoelectric material having vinylidene fluoride as a main component, and the organic piezoelectric material has been heat treated and has a relative dielectric constant in a thickness resonance frequency being 10 or more.

[2] The oscillator for ultrasonic wave reception of item 1 above, wherein the organic piezoelectric material is a stretched film.

[3] The oscillator for ultrasonic wave reception of item 1 or 2 above, wherein the organic piezoelectric material has been subjected to intermolecular cross-linking.

[4] The oscillator for ultrasonic wave reception of any one of items 1 through 3 above, wherein the intermolecular cross-linking of the organic piezoelectric material has been carried out by electron beam irradiation before polarization treatment is carried out.

[5] The oscillator for ultrasonic wave reception of any one of claims 1 through 4, wherein the electron beam irradiation amount of the election beam irradiation in the organic piezoelectric material is from 0.1 to 50 kGy.

[6] The oscillator for ultrasonic wave reception of any one of items 1 through 5 above, wherein the organic piezoelectric material contains a cross-linking agent having two or more functional groups.

[7] A manufacturing method of the oscillator for ultrasonic wave reception of any one of items 1 through 6 above, wherein polarization treatment is carried out before an electrode, which is to be formed on both sides of the organic piezoelectric material film, is formed, after the electrode has been formed only on one side of the organic piezoelectric material film, or after the electrode has been formed on both sides of the organic piezoelectric material film.

[8] The manufacturing method of the oscillator for ultrasonic wave reception of item 7 above, wherein the polarization treatment is voltage application treatment

[9] An ultrasonic wave probe comprising an oscillator for ultrasonic wave transmission and an oscillator for ultrasonic wave reception, wherein the oscillator for ultrasonic wave reception is the oscillator for ultrasonic wave reception of any one of claims 1 through 6.

[10] The ultrasonic wave probe of claim 9, comprising the oscillator for ultrasonic wave reception of any one of items 1 through 6 above, wherein the oscillator for ultrasonic wave reception is a laminated oscillator composed of not less than two layers, including the organic piezoelectric material laminated with a polymeric material different from the organic piezoelectric material, and the thickness of the laminated oscillator is from 40 to 150 μm.

[11] An ultrasonic wave medical diagnostic imaging system comprising an electric signal generating device, an ultrasonic wave probe in which a plurality of oscillators are arranged which receive the electric signal and transmit an ultrasonic wave to an examinee and generate a reception signal according to a reflection wave returned from the examinee and an image processing device which forms an image of the examinee according to the reception signal generated by the ultrasonic wave probe, wherein the ultrasonic wave probe comprises the oscillator for ultrasonic wave reception of any one of items 1 through 6 above.

### Effects of the Invention

The above means of the invention can provide an oscillator for ultrasonic wave reception which is excellent in piezoelectric characteristic and thermal resistance and is suitable for high frequency and broad band, a manufacturing method of the oscillator, an ultrasonic wave probe employing the oscillator, and an ultrasonic wave medical diagnostic imaging system.

### BRIEF DESCRIPTION OF THE DRAWING

FIG. 1 is a schematic drawing showing the structure of the main section of an ultrasonic wave medical diagnostic imaging system.

### EXPLANATION OF TILE SYMBOLS

1. Piezoelectric material for reception
2. Support
3. Piezoelectric material for transmission
4. Backing layer
5. Electrode
6. Acoustic lens

### PREFERRED EMBODIMENT FOR CARRYING OUT THE INVENTION

The oscillator for ultrasonic wave reception is used in a probe for an ultrasonic wave medical diagnostic imaging system and is one comprising a piezoelectric material for ultrasonic wave reception, characterized in that the piezoelectric material for ultrasonic wave reception is an organic piezoelectric material having vinylidene fluoride as a main component, and the organic piezoelectric material has been heat treated and has a relative dielectric constant in a thickness resonance frequency being 10 or more. This characteristic is a technical characteristic common through items 2 through 11 described above.

Herein, “a thickness resonance frequency” refers to a thickness resonance frequency in the mode oscillating in the thickness direction of an oscillator in the form of film. In the invention, “a relative dielectric constant” refers to a relative dielectric constant in the thickness resonance frequency obtained by measuring a sample with electrodes on both sides at 25° C. through an impedance analyzer, in which the electrodes are connected to each other by a lead. The relative dielectric constant is preferably from 10 to 100, and more preferably from 10 to 50.

In the embodiment of the invention, the organic piezoelectric material is preferably formed into a film and stretched, in view of piezoelectric characteristic. The heat treatment is carried out preferably at a temperature at least 10° C. lower than the melting point of the organic piezoelectric material. It is preferred that the organic piezoelectric material has been intermolecularly cross-linked, in view of piezoelectric characteristic and thermal resistance. As the intermolecular cross-linking method, there can be used various conventional methods, however, it is preferred that the intermolecular cross-linking of the organic piezoelectric material has been carried out by electron beam irradiation before polarization treatment. It is preferred that the electron beam irradiation amount in the electron beam irradiation is from 0.1 to 50 kGy. It is preferred that a cross-linking agent having not less than two functional groups is contained in the organic piezoelectric material.

In the manufacturing method of the oscillator for ultrasonic wave reception, it is preferred that polarization treatment is carried out before an electrode, which is to be formed on both sides of the organic piezoelectric material film, is formed, after the electrode has been formed only on one side of the organic piezoelectric material film, or after the electrode has been formed on both sides of the organic piezoelectric material film. The polarization treatment is preferably a voltage application treatment.

The oscillator for ultrasonic wave reception of the invention can constitute an ultrasonic wave probe together with an oscillator for ultrasonic wave transmission. In this case, it is preferred that the ultrasonic wave probe comprises the oscillator for ultrasonic wave reception of the invention which is a laminate oscillator comprising an organic piezoelectric material and composed of not less than two layers, the organic piezoelectric material laminated with a polymeric material different from the organic piezoelectric material, and the thickness of the laminate oscillator is from 40 to 150 μm.

The oscillator for ultrasonic wave reception of the invention or the ultrasonic wave probe employing the same can be suitably applied to an ultrasonic wave medical diagnostic imaging system

Next, the invention, its constituent and the preferred embodiment of the invention will be explained in detail below.

### (Oscillator for Ultrasonic Wave Transmission and Reception)

The oscillator for ultrasonic wave reception of the invention is characterized in that it is an oscillator for ultrasonic wave reception used in a probe for an ultrasonic wave medical diagnostic imaging system equipped with an oscillator for ultrasonic wave transmission and an oscillator for ultrasonic wave transmission.

Generally, an ultrasonic wave oscillator has a structure that a layer (or film) formed from a piezoelectric material in the form of a film (hereinafter also referred to as a piezoelectric material layer or a piezoelectric material film) is inserted between a pair of electrodes. A plurality of ultrasonic wave oscillators are arranged, for example, one dimensionally, thereby obtaining an ultrasonic wave probe.

The probe has a function of driving a specific number of oscillators in the longitudinal direction in the plurality of ultrasonic wave oscillators arranged to irradiate the site to be examined in an examinee with convergent ultrasonic wave beams, receive the ultrasonic wave reflection echo returned from the site, and convert the echo to an electric signal.

Next, the oscillator for ultrasonic wave reception of the invention and the oscillator for ultrasonic wave transmission will be explained in detail, respectively.

### (Oscillator for Ultrasonic Wave Reception)

The oscillator for ultrasonic wave reception of the invention is used in a probe for an ultrasonic wave medical diagnostic imaging system and is an oscillator comprising a piezoelectric material for ultrasonic wave reception, characterized in that the piezoelectric material is an organic piezoelectric material having vinylidene fluoride as a main component and the organic piezoelectric material has been subjected to electron beam irradiation and has a relative dielectric constant in a thickness resonance frequency being from 10 to 50.

In the embodiment of the invention, the organic piezoelectric material is preferably formed into a film and stretched.

It is preferred that the electron beam irradiation is carried out before polarization treatment is carried out When the organic piezoelectric material is irradiated with an electron beam, the electron beam irradiation amount is preferably from 0.1 to 50 kGy in view of flexibility and piezoelectric characteristic.

Further, the organic piezoelectric material preferably contains a cross-linking agent having two or more functional groups.

### <<Organic Piezoelectric Material Constituting Piezoelectric Material for Reception>>

A material of the piezoelectric material for reception constituting the oscillator for ultrasonic wave reception of the invention is a polymeric material having vinylidene fluoride as a main component, in view of good piezoelectric characteristic or availability.

Specifically, the polymeric material is required to be a homopolymer of vinylidene fluoride or a copolymer containing vinylidene fluoride as a main component, each containing a CF₂ group exhibiting a large dipole moment. Examples of a component in the copolymer other than vinylidene fluoride include tetrafluoroethylene, trifluoroethylene, hexafluoropropane and chlorofluoroethylene.

For example, in a vinylidene fluoride/trifluoroethylene copolymer, the electric mechanical combination constant (piezoelectric effect) in the thickness direction varies due to the copolymerization ratio and therefore, the copolymerization ratio of the vinylidene fluoride in the copolymer is preferably from 60 to 99 mol %, and more preferably from 85 to 99 mol %.

A copolymer of 85-99 mol % of vinylidene fluoride and 1-15 mol % of perfluoroalkyl vinyl ether, perfluoroalkoxyethylene or perfluorohexaethylene restrains a transmission basic wave and results in enhanced sensitivity to a harmonic component reception in combination of an inorganic piezoelectric element for transmission and an organic piezoelectric element for reception.

The polymer piezoelectric material described above can be formed into a thin film as compared with an inorganic piezoelectric material formed from ceramics and can provide an oscillator applied to reception and transmission of higher frequency.

In the invention, the organic piezoelectric material is characterized in that it has a relative dielectric constant in the thickness resonance frequency of from 10 to 50. Adjustment of the relative dielectric constant can be carried out by adjustment of the number of a polar functional group such as a CF₂ group or a CN group contained in a compound constituting the organic piezoelectric material, the composition or the polymerization degree or by polarization treatment described later.

The organic piezoelectric material film constituting the oscillator for reception of the invention can be a laminate in which a plurality of polymeric materials are multi-layered. In addition to the polymeric materials described above, polymeric materials having a relatively low relative dielectric constant as shown below can be used in combination as the polymeric materials to be multi-layered in the laminate.

In the following examples, the figures in the parentheses represent a relative dielectric constant of the polymeric materials (resins).

Examples of the polymeric materials include methyl methacrylate resin (3.0), acrylonitrile resin (4.0), acetate resin (3.4), aniline resin (3.5), aniline formaldehyde resin (4.0), aminoalkyl resin (4.0), alkyd resin (5.0), nylon 6-6 (3.4), ethylene resin (2.2), epoxy resin (2.5), vinyl chloride vinylidene chloride resin (3.0), urea formaldehyde resin (7.0), polyacetal resin (3.6), polyurethane (5.0), polyester resin (2.8), polyethylene (low pressure) (2.3), polyethylene terephthalate (2.9), polycarbonate resin (2.9), melamine resin (5.1), melamine formaldehyde resin (8.0), cellulose acetate (3.2), vinyl acetate resin (2.7), styrene resin (2.3), styrene butadiene rubber (3.9) and ethylene fluoride resin (2.0).

It is preferred that the polymeric materials having a low relative dielectric constant as shown above are properly selected according to various objects, for example, for the purpose of adjusting the piezoelectric characteristic or increasing physical strength of the organic piezoelectric material layer.

### (Oscillator for Ultrasonic Wave Transmission)

The oscillator for ultrasonic wave transmission in the invention is preferably formed from a piezoelectric material having an appropriate relative dielectric constant in connection with the oscillator having the piezoelectric material for reception as described above. A piezoelectric material having excellent thermal resistance and voltage resistance is preferably employed.

As materials for constituting the oscillator for ultrasonic wave transmission, various known organic or inorganic piezoelectric materials can be employed.

As the organic piezoelectric materials, there can be employed the same as the polymeric materials described above in the organic piezoelectric materials constituting the oscillator for ultrasonic wave reception.

Examples of the inorganic piezoelectric materials include quartz, lithium niobate (LiNbO₃), potassium niobate tantalate [K(Ta, Nb)O₃], barium titanate, (BaTiO₃), lithium tantalite (LiTaO₃), lead titanate zirconate (PZT), strontium titanate (SrTiO₃) and barium strontium titanate (BST). PZT is preferably Pb(Zr₁₋ₙTiₙ)O₃ (0.47≦n≦1).

### (Manufacturing Method of Organic Piezoelectric Material Layer)

The organic piezoelectric material layer in the invention can be manufactured according to the various methods, employing the above polymeric material as a main constituent

As a manufacturing method of the organic piezoelectric material layer, there is a method which forms a polymer layer by coating a solution of the above polymeric materials on a substrate and drying the coated layer or a method which forms a polymer layer on a substrate according to a known vapor deposition polymerization method or a solution polymerization coating method, each employing a raw material of the above polymeric materials.

With respect to the typical method and conditions of the vapor deposition polymerization method, those disclosed in Japanese Patent O.P.I. Publication Nos. 7-258370, 5-311399 and 2006-49418 are referred to.

With respect to the typical method and conditions of the solution polymerization coating method_{;} known various ones can be applied. For example, a method is preferred which comprises the steps of coating a mixed solution of raw materials on a substrate, drying the coated solution (removing the solvent) to some extent under reduced pressure, then carrying out thermal polymerization, polarizing treatment being carried out at the same time when or after the thermal polymerization is carried out, thereby obtaining an organic piezoelectric material film.

### <<Film Formation and Stretching>>

When the organic piezoelectric material having vinylidene fluoride in the invention is used as an oscillator, generally, the material is formed into a film, and provided with a surface electrode for inputting an electric signal.

The film formation can be carried out according to a general method such as a melting method or a casting method. It is known that a vinylidene fluoride-trifluoroethylene copolymer film itself can form a crystal having polarity. In order to further improve the characteristics, treatment for arranging the molecular alignment is required. Examples of the treatment include stretching treatment and polarization treatment.

As the stretching methods, various known methods can be employed. For example, a solution in which the polymeric materials described above are dissolved in an organic solvent such as methyl ethyl ketone (MEK) is cast on a substrate such as a glass plate, dried by evaporating the solvent at ordinary temperature to obtain a film with a predetermined thickness, and stretched by a predetermined factor at room temperature. The stretching can be carried out mono-axially or bi-axially, provided that the organic piezoelectric material film in a predetermined form is not destroyed. The stretching factor is preferably from 2 to 10, and preferably from 2 to 6.

In a vinylidene fluoride-trifluoroethylene copolymer and/or a vinylidene fluoride-tetrafluoroethylene copolymer, the use of a polymeric piezoelectric material exhibiting a melt flow rate of not more than 0.3 g/min at 230° C., preferably not more than 0.02 g/min and more preferably not more than 0.01 g/min can provide a highly sensitive piezoelectric material thin layer.

### <<Heat Treatment>>

In the invention, it is preferred that the organic piezoelectric material has been heat treated. As the heat treatment method of the organic piezoelectric material in the invention, a method is preferred in which a film of the organic piezoelectric material is allowed to stand around at a temperature, the upper limit of which is 10° C. lower than the melting point of the film while holding the both ends of the film by a chuck or a clip, in order to apply heat effectively or uniformly to the inside of the film. A method which heats the film by bringing the film in direct contact with a heat source such as a heated plate is undesired, since a material which causes contraction on heat application impairs planarity of the film. Rather, it is effective in securing the planarity to conduct relaxation treatment against the contraction caused on heat application. The relaxation treatment herein referred to means one which varies stress applied to the both ends of the film while following contraction or expansion force to which the film is subjected in the process in which the heat treatment is carried out, followed by cooling to room temperature. The relaxation treatment, as long as the planarity of the film is not impaired by film relaxation or the breakage of the film does not occur due to too much stress, may be conducted to contract the film by relaxation of stress or to broaden the film in the direction applying tensile force so as not to cause stretching. The organic piezoelectric material containing polyvinylidene fluoride as a main component has a melting point of from 150 to 180° C., and therefore, it is preferred that it is heat treated at 100 to 140° C. The effect is seen when the heat treatment is carried out for 30 minutes or more, and the longer the heat treatment time is, the more the crystal growth promotes. Since the crystal growth saturates with time, the heat, treatment time is actually from 1 hour to around 10 hours, and at most 24 hours.

### <<Electron Beam Irradiation>>

The invention is characterized in that in order to solve the low phase transition temperature of polyvinylidene fluoride, the organic piezoelectric material is intermolecularly cross-linked by electron beam irradiation in the state in which the molecules are aligned in order. The appropriate irradiation amount of the electron beam causes intramolecular cleavage, resulting in intermolecular cross-linking with release of hydrogen fluoride.

In order to promote the intermolecular cross-linking, a polyfunctional cross-linking agent having two or more functional groups can be added. As the cross-linking agent, for example, a triallyl isocyanate with a functional group such as an isocyanato group can be used.

Generally, when a fluorine-containing polymer is subjected to electron beam irradiation, intramolecular cleavage preferentially occurs. Therefore, mechanical properties of the materials obtained lowers. In the invention, when the material in the molecular alignment state after stretching and prior to polarization treatment is subjected to an electron beam irradiation of from 0.1 to 50 kGy, thermal resistance is increased without impairing physical properties.

### <<Polarization Treatment>>

As a polarization treatment method in the polarization treatment in the invention, there can be applied a method according to a well-known direct current voltage application treatment, alternating current voltage application treatment, or corona discharge treatment.

For example, the corona discharge treatment in the corona discharge treatment method can be carried out employing an apparatus available on the market composed of a high voltage power source and an electrode.

It is preferred that discharge conditions are properly selected, since they vary due to kind of an apparatus used or treatment ambience. When the high voltage power source is used, the voltage is preferably from −1 to −20 kV, the current is preferably from 1 to 80 mA, the distance between the electrodes is preferably from 1 to 10 cm, and voltage applied is preferably from 0.5 to 2.0 MV/m.

The electrode used is preferably a needle electrode, a linear electrode (wire electrode) or a network electrode, each being conventionally used, but the invention is not limited thereto.

### (Substrate)

The substrate used is selected according to usage of the organic piezoelectric material layer in the invention. As the substrate in the invention there can be used a plate or film of a plastic such as polyimide, polyamide, polyimideamide, polyethylene terephthalate (PET), polyethylene naphthalate (PEN), polymethyl methacrylate (PMMA), a polycarbonate resin, or a cycloolefin polymer. The substrate may be those in which the surface of these materials is covered with aluminum, gold, copper, magnesium, or silicon. The substrate may be a plate or film of aluminum, gold, copper, magnesium, silicon or a single crystal of a halide of rare earth element.

### (Electrode)

The oscillator comprising a piezoelectric material in the invention is one which is manufactured by forming an electrode on one or both sides of a piezoelectric material film (layer), and subjecting the piezoelectric material layer to polarization treatment. The electrode is formed from electrode materials comprised mainly of gold (Au), platinum (Pt), silver (Ag), palladium (Pd), copper (Cu), nickel (Ni), or tin (Sn).

In the formation of the electrode, a layer of a metal such as titanium (Ti) or chromium (Cr) is formed according to a sputtering method as an under layer to obtain a thickness of from 0.02 to 1.0 μm, and metal materials composed mainly of the metal elements described above or metal materials composed of alloys thereof, and optionally insulation materials are deposited on the under layer according to a sputtering method or another appropriate method to form a 1 to 10 μm thick layer. The electrode formation can be carried out by a screen printing method employing a conductive paste in which fine metal particles are mixed with a low melting point glass, a dipping method or a melt splaying method.

Further, a given voltage is applied across electrodes formed on both sides of a piezoelectric material layer to polarize the piezoelectric material layer. Thus, a piezoelectric element is obtained.

### (Ultrasonic Wave Probe)

The ultrasonic wave probe of the invention is a probe for a medical diagnostic imaging system comprising an oscillator for ultrasonic wave transmission and an oscillator for ultrasonic wave reception, and the invention is characterized in that the oscillator for ultrasonic wave reception of the invention described above is employed as an oscillator for reception.

In the invention, one oscillator may bear both of ultrasonic wave transmission and reception, but it is preferred that an oscillator for ultrasonic wave transmission and an oscillator for ultrasonic wave reception are separately provided in a probe.

A piezoelectric material constituting an oscillator for ultrasonic wave transmission may be a conventional ceramics inorganic piezoelectric material or an organic piezoelectric material.

In the ultrasonic wave probe of the invention, the oscillator for ultrasonic wave reception of the invention can be disposed on an oscillator for transmission or in parallel.

Preferred embodiment is one having a structure that the oscillator for ultrasonic wave reception of the invention is provided on an oscillator for ultrasonic wave transmission. In this case, the oscillator for ultrasonic wave reception of the invention, which is laminated on another polymeric material (a film as a substrate of the polymer (resin) having a relatively low relative dielectric constant as described above, for example, a polyester film), may be provided on the oscillator for ultrasonic wave transmission. It is preferred that the total thickness of the laminate of the oscillator and the polymeric material matches a preferable reception frequency band region in view of design of the probe. The thickness is preferably from 40 to 150 μm in view of an ultrasonic wave medical diagnostic imaging system for practical use or actual frequency band used for collection of living body information.

The probe may be provided with a backing layer, an acoustic matching layer, an acoustic lens and the like. The probe may be one in which many oscillators having a piezoelectric material are two dimensionally arranged. The plural probes, being two dimensionally arranged, may constitute a scanner in which the plural probes conduct scanning in order, followed by imaging.

### (Ultrasonic Wave Medical Diagnostic Imaging System)

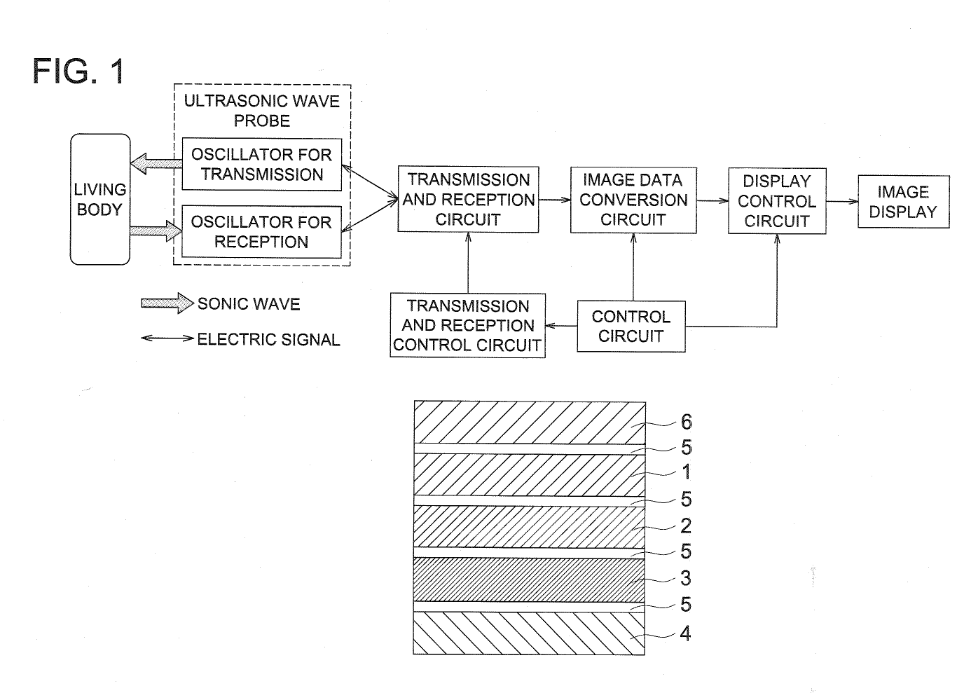

The above ultrasonic wave probe of the invention can be applied to various ultrasonic wave diagnostic systems. For example, it can be suitably applied to an ultrasonic wave medical diagnostic imaging system as shown in FIG. 1.

FIG. 1 is a schematic drawing showing the structure of the main section of an ultrasonic wave medical diagnostic imaging system in the embodiment of the invention. The ultrasonic wave medical diagnostic imaging system is equipped with an ultrasonic wave probe (probe) in which a piezoelectric material oscillator is arranged which transmits an ultrasonic wave to an examinee such as a patient and receives an ultrasonic wave reflected from the examinee as an echo signal. The ultrasonic wave medical diagnostic imaging system is further equipped with a transmission and reception circuit, which supplies an electric signal to the ultrasonic wave probe to generate ultrasonic wave and receives an echo signal which each piezoelectric material oscillator in the ultrasonic wave probe receives, and a transmission and reception control circuit, which controls transmission and reception of the transmission and reception circuit.

The system is further equipped with an image data conversion circuit which converts an echo signal which the transmission and reception circuit receives to an ultrasonic wave image data of an examinee. The system is equipped with a display control circuit, which controls a monitor with an ultrasonic wave image data converted by the image data conversion circuit and displays an image, and a control circuit, which controls the entire ultrasonic wave medical diagnostic imaging system.

The transmission and reception control circuit, the image data conversion circuit and the display control circuit are connected to the control circuit and the operation thereof is controlled through the control circuit. An electric signal is applied to each piezoelectric oscillator in the ultrasonic wave probe to transmit an ultrasonic wave to an examinee and a reflection wave generated by acoustic impedance mismatch inside the examinee is received by the ultrasonic wave probe.

The transmission and reception circuit described above corresponds to “an electric signal generation means”, and the image data conversion circuit corresponds to “an image processing means”.

The ultrasonic wave diagnostic system as described above, comprising the oscillator for ultrasonic wave reception of the invention which is excellent in piezoelectric characteristic and thermal resistance and is suitable for high frequency and broad band, can provide an ultrasonic wave image with improved image quality and reproduction stability as compared with a conventional one.

### Examples

Next, the present invention will be explained employing examples, but the invention is not limited thereto.

### (Manufacture and Evaluation of Organic Piezoelectric Material Film)

### Example 1

Powder of a vinylidene fluoride copolymer having a vinylidene fluoride (hereinafter also referred to as VDF)/trifluoroethylene (hereinafter also referred to as 3FE) ratio of 80:20 was dissolved in a 50° C. methyl ethyl ketone (hereinafter also referred to as MEK), cast on a glass plate, and dried by evaporating the solvent at an ordinary temperature to obtain a film (organic piezoelectric material film) with a thickness of about 140 μm. The resulting film was stretched by a factor of four at room temperature, and then heat treated at 135° C. for 1 hour while maintaining the stretched length. The endothermic peak temperatures of the thus obtained heat treated film were 120° C. and 153° C.

Subsequently, the film was irradiated with 0.1 Mrad of electron beam accelerated at an acceleration voltage of 250 kV, employing an electron beam accelerator. Thereafter, gold/aluminum was evaporation deposited on both sides of the resulting film to provide a surface resistance of not more than 1Ω. Thus, a sample with electrodes on the surface was obtained.

The resulting sample was subjected to polarization treatment, while an alternating voltage of 0.1 Hz is applied to the electrodes. The polarization treatment was carried out while gradually increasing voltage from a low voltage to a final electric field between the two electrodes being 50 MV/m.

### Example 2

Film with electrodes was manufactured in the same manner as in Example 1, except that triallyl isocyanate was further added to the above copolymer MEK solution in an amount of 1% based on the weight of the copolymer dissolving in the copolymer MEK solution. Thus, Sample 2, which was subjected to polarization treatment, was obtained.

### Examples 3 and 4, and Comparative Examples

Films with electrodes were manufactured in a similar manner as in Example 1 or 2, provided that heat treatment, stretch treatment, electron beam irradiation and cross-linking agent addition as shown in Table 1 were carried out. Thus, samples, which were subjected to polarization treatment, were obtained.

### [Evaluation Method of Organic Piezoelectric Material Film]

The electrodes on both sides of each of the above-obtained samples with electrodes being connected by a lead, each sample was scanned at 25° C. with 600 frequencies of the same interval in the frequency range of from 40 Hz to 110 MHz, employing an impedance analyzer 4294A manufactured by Agilent Technologies, Inc. The relative dielectric constant at the thickness resonance frequency was determined. Similarly, a peak frequency P of resistance and a peak frequency S of conductance approximately at the thickness resonance frequency were determined, and electric mechanical combination constant kₜ was determined according to the following equation.

kₜ=(α/tan(α))^{1/2}

wherein α=(π/2)×(S/P)

A method of determining the electric mechanical combination constant from the thickness resonance frequency employing an impedance analyzer is in accordance with item 4.6.6 Thickness longitudinal Oscillation of Disk-shaped Oscillator described in electrical test method of piezoelectric ceramic oscillator in Japan Electronics and Information Technology Industries Association Standard JEITA EM-4501 (formerly, EMAS-6100).

After kₜ had been determined, the sample was put into an oil bath heated at 180° C. for 10 minutes, and then taken out from the bath. After the oil of the sample was wiped off, electric mechanical combination constant kₜ′ of the heated sample was determined in the same manner as above. The value (kₜ′/k)×100 was determined as a measure of thermal resistance. The nearer to 100 the value is, the higher the thermal resistance.

The evaluation results are shown in Table 1.

TABLE 1

| | a | b | c | d | e | f | g | Remarks |
|---|---|---|---|---|---|---|---|---|
| Ex. 1 | Yes | Yes | 0.1 | None | 23 | 0.33 | 99 | — |
| Ex. 2 | Yes | Yes | 10 | 1.0 | 20 | 0.31 | 100 | — |
| Ex. 3 | Yes | Yes | 10 | 1.5 | 18 | 0.31 | 100 | — |
| Ex. 4 | Yes | Yes | 50 | 1.5 | 15 | 0.28 | 97 | — |
| Comp. Ex. 1 | Yes | Yes | None | None | 20 | 0.33 | 30 | — |
| Comp. Ex. 2 | Yes | Yes | 100 | 1.0 | 7 | — | — | fragile |
| Comp. Ex. 3 | No | No | 10 | 1.0 | 5 | 0.21 | 95 | — |
| Comp. Ex. 4 | No | No | None | None | 5 | 0.12 | 30 | — |

a: Heat treatment,
b: Stretch treatment,
c: Electron beam irradiation Mrad,
d: Cross-linking agent (%),
e: Relative dielectric constant εᵣ,
f: Piezoelectric characteristic kₜ,
g: Thermal resistance kₜ′/kₜ (%)

As is apparent from Table 1, inventive samples have high relative dielectric constant and excellent piezoelectric characteristic, and particularly exhibit excellent thermal resistance that does not deteriorate in the thermal resistance test. Accordingly, it is considered that the inventive samples do not lower the performances due to heat generation during processing, heat generation during employing as a probe, or any other heat generation.

### (Preparation and Evaluation of Probe)

### Example 5

### (Preparation of Piezoelectric Material for Transmission)

CaCO₃, La₂O₃, Bi₂O₃ and TiO₂ were provided as component materials, and MnO as a subcomponent material. The component materials were weighed so that a final component composition was (Ca_{0.97}La_{0.03})Bi_{4.01}Ti₄O₁₅. Subsequently, the materials were added with water, mixed for 8 hours in a ball mill charged with media made of zirconia, and then sufficiently dried to obtain a mixture powder. The resulting mixture powder was temporarily molded and subjected to temporary calcination in air at 800° C. for 2 hours to obtain a preliminary calcination product. Subsequently, the preliminary calcination product was added with water, pulverized in a ball mill charged with media made of zirconia, and then dried to obtain a piezoelectric ceramics material powder. The pulverization time and the pulverization conditions during the pulverization being changed, a piezoelectric ceramics material powder having a particle size of 100 nm was obtained. The piezoelectric ceramics material powder having a different particle size was added with 6% by weight of water as a binder, and press molded to obtain a preliminary plate-like molding having a thickness of 100 μm. The resulting preliminary plate-like molding was subjected to calcination to obtain a calcination product having a thickness of 20 μm as a final calcination product. The calcination temperature was 1100° C. An electric field of not less than 1.5× Ec (MV/m) being applied, the calcination product was subjected to polarization treatment.

### (Preparation of Laminate Oscillator for Reception)

The vinylidene fluoride copolymer film (organic piezoelectric material film) subjected to electron beam irradiation obtained in Example 1 was adhered to a 50 μm thick polyester film through an epoxy adhesive to obtain a laminate oscillator. The resulting laminate oscillator was further subjected to polarization treatment in the same manner as above.

Subsequently, the resulting laminate oscillator for reception was laminated on the piezoelectric material for transmission described above according to an ordinary method, and further provided with a backing layer and an acoustic consistency layer. Thus, an ultrasonic wave probe was prepared.

An ultrasonic wave probe for comparison was prepared in the same manner as the ultrasonic wave probe obtained above, except that a laminate oscillator employing only the vinylidene fluoride copolymer film (organic piezoelectric material film) was used instead of the laminate oscillator for reception.

Subsequently, the two ultrasonic wave probes obtained above were evaluated for reception sensitivity and dielectric breakdown strength.

With regard to the reception sensitivity, a fundamental frequency f1 of 5 MHz was transmitted, and then, relative reception sensitivity of a reception secondary harmonic f₂ of 10 MHz, a reception tertiary harmonic f₃ of 15 MHz and a reception quaternary harmonic f₄ of 20 MHz was determined. The relative reception sensitivity was measured, employing a sound intensity measuring system Model 805 (1 to 50 MHz), manufactured by Sonora Medical System, Inc., 2021 Miller Drive Longmont, Colo. (0501 USA).

After the above probes were subjected to load test in which a load power increased to five times was applied for 10 hours, relative reception sensitivity of the resulting probes was measured and evaluated as a measure of the dielectric breakdown strength. Sensitivity, lowering by not more than 1% of that before subjected to the load test, was evaluated as good. Sensitivity, lowering by less than 10% to more than 1% of that before subjected to the load test, was evaluated as accepted. Sensitivity, lowering by not less than 10% of that before subjected to the load test, was evaluated as unacceptable.

In the above evaluation, it proved that the probe with the reception piezoelectric (material) laminate oscillator of the invention had relative reception sensitivity about 1.2 times that of the probe for comparison, and had high dielectric breakdown strength. That is, it was confirmed that the oscillator for ultrasonic wave reception of the invention was suitably applied to a probe used in the ultrasonic wave medical diagnostic imaging system as shown in FIG. 1.

1. An oscillator for ultrasonic wave reception used in a probe for an ultrasonic wave medical diagnostic imaging system, the oscillator for ultrasonic wave reception comprising a piezoelectric material for ultrasonic wave reception, wherein the piezoelectric material for ultrasonic wave reception is an organic piezoelectric material having vinylidene fluoride as a main component, and the organic piezoelectric material has been heat treated and has a relative dielectric constant in a thickness resonance frequency being 10 or more.

2. The oscillator for ultrasonic wave reception of claim 1, wherein the organic piezoelectric material is a stretched film.

3. The oscillator for ultrasonic wave reception of claim 1, wherein the organic piezoelectric material has been subjected to intermolecular cross-linking.

4. The oscillator for ultrasonic wave reception of claim 3, wherein the intermolecular cross-linking of the organic piezoelectric material has been carried out by electron beam irradiation before polarization treatment is carried out.

5. The oscillator for ultrasonic wave reception of claim 4, wherein the electron beam irradiation amount of the electron beam irradiation in the organic piezoelectric material is from 0.1 to 50 kGy.

6. The oscillator for ultrasonic wave reception of claim 1, wherein the organic piezoelectric material contains a cross-linking agent having two or more functional groups.

7. (canceled)

8. The manufacturing method of the oscillator for ultrasonic wave reception of claim 12, wherein the polarization treatment is voltage application treatment.

9. An ultrasonic wave probe comprising an oscillator for ultrasonic wave transmission and an oscillator for ultrasonic wave reception, wherein the oscillator for ultrasonic wave reception comprises a piezoelectric material for ultrasonic wave reception, wherein the piezoelectric material for ultrasonic wave reception is an organic piezoelectric material having vinylidene fluoride as a main component, and the organic piezoelectric material has been heat treated and has a relative dielectric constant in a thickness resonance frequency being 10 or more.

10. The ultrasonic wave probe of claim 9, wherein the oscillator for ultrasonic wave reception is a laminated oscillator composed of not less than two layers, including the organic piezoelectric material laminated with a polymeric material different from the organic piezoelectric material, and the thickness of the laminated oscillator is from 40 to 150 μm.

11. An ultrasonic wave medical diagnostic imaging system comprising an electric signal generating device, an ultrasonic wave probe in which a plurality of oscillators are arranged which receive the electric signal and transmit an ultrasonic wave to an examinee and generate a reception signal according to a reflection wave returned from the examinee and an image processing device which forms an image of the examinee according to the reception signal generated by the ultrasonic wave probe, wherein the ultrasonic wave probe comprises an oscillator for ultrasonic wave reception comprising a piezoelectric material for ultrasonic wave reception, wherein the piezoelectric material for ultrasonic wave reception is an organic piezoelectric material having vinylidene fluoride as a main component, and the organic piezoelectric material has been heat treated and has a relative dielectric constant in a thickness resonance frequency being 10 or more.

12. A manufacturing method of an oscillator for ultrasonic wave reception used in a probe for an ultrasonic wave medical diagnostic imaging system, the oscillator for ultrasonic wave reception comprising a piezoelectric material film for ultrasonic wave reception and an electrode provided on both sides of the piezoelectric material film, wherein the piezoelectric material for ultrasonic wave reception is an organic piezoelectric material having vinylidene fluoride as a main component, and the organic piezoelectric material has been heat treated and has a relative dielectric constant in a thickness resonance frequency being 10 or more, the manufacturing method comprising the step of:
carrying out polarization treatment, before the electrode is formed, after the electrode has been formed on one side of the organic piezoelectric material film, or after the electrode has been formed on both sides of the organic piezoelectric material film.
